(19) United States
(12) Patent Application Publication
(10) Pub. No.: US 2025/0009377 A1
(43) Pub. Date: Jan. 9, 2025

(54) FORCEPS DEVICE

(71) Applicant: RIVERFIELD INC., Tokyo (JP)

(72) Inventor: Kazuki AKIYOSHI, Tokyo (JP)

(73) Assignee: RIVERFIELD INC., Tokyo (JP)

(21) Appl. No.: 18/890,956

(22) Filed: Sep. 20, 2024

Related U.S. Application Data
(63) Continuation of application No. PCT/JP2022/013610, filed on Mar. 23, 2022.

(51) Int. Cl. A61B 17/29

(52) U.S. Cl. CPC A61B 17/29; A61B 2017/2932

(57) ABSTRACT

A forceps device includes a distal end part including a treatment part for treating an object, a base cap connected proximally to the distal end part, a pipe connected proximally to the base cap, a cover that covers the distal end part and the base cap and has an end face at a proximal end of the base cap, and an enlarged-diameter portion having a diameter that is larger than a diameter of the end face of the cover.

### CROSS REFERENCE TO RELATED APPLICATION

This application is a continuation application of International Patent Application No. PCT/JP2022/013610 filed on Mar. 23, 2022, in the Japan Patent Office, the contents of which being incorporated by reference herein in its entirety.

### BACKGROUND

The present disclosure relates to a forceps device.

Medical treatments using robots (manipulators) have recently been proposed in order to reduce the burden on operators and save manpower in medical facilities. In the field of surgery, proposals have been made for surgical manipulator systems for operators to treat patients by operating remotely-controllable surgical manipulators.

Various surgical tools may be attached to distal ends of surgical manipulators. For example, the surgical tools may include forceps for grasping human tissue during surgery. Some surgical tools may have a cover.

### SUMMARY

It is an aspect to provide a novel technology that makes a cover less likely to come off a surgical tool or move from a correct position of the cover.

According to an aspect of one or more embodiments, there is provided a forceps device comprising a distal end part including a treatment part for treating an object; a base cap connected proximally to the distal end part; a pipe connected proximally to the base cap; a cover that covers the distal end part and the base cap and has an end face at a proximal end of the base cap; and an enlarged-diameter portion having a diameter that is larger than a diameter of the end face of the cover.

According to another aspect of one or more embodiments, there is provided a forceps device comprising a distal end part including a treatment part for treating an object; a base cap connected proximally to the distal end part; a pipe connected proximally to the base cap; a cover that covers the distal end part and the base cap and has an end face at a proximal end of the base cap; and an enlarged-diameter portion having a diameter that is larger than a diameter of the end face of the cover.

According to yet another aspect of one or more embodiments, there is provided a forceps device comprising a distal end part including a treatment part for treating an object; a base cap connected proximally to the distal end part; a pipe connected proximally to the base cap; a cover that covers the distal end part and the base cap and has an end face at a proximal end of the base cap; and a hood having a diameter that is larger than a diameter of the end face of the cover.

### BRIEF DESCRIPTION OF DRAWINGS

FIG. 1 is a front view of a forceps device according to an embodiment.

FIGS. 2A and 2B are schematic views for explaining a state in which a joint cover comes off a base cap, according to some embodiments.

FIG. 3 is an enlarged view of a structure around an enlarged-diameter portion of the forceps device, according to some embodiments.

FIG. 4 is a schematic view for explaining a difference in deflection depending on a position at which an end of a trocar comes in contact with the joint cover, according to some embodiments.

### DETAILED DESCRIPTION

As described above, a surgical tool may have a cover. For example, the cover for a surgical treatment tool may be a member having a cylindrical shape to surround at least a base of the surgical treatment tool. In some cases, the cover for a surgical treatment tool may be made of an insulating material.

In some cases, the cover for a surgical treatment tool, however, is made of a flexible material such as rubber, which can be used during endoscopic surgery or the like. Thus, when the surgical treatment tool is inserted or removed through a trocar, the cover may get caught on an end of the trocar. Thus, in some cases, the cover may come off the surgical treatment tool or may be moved from its correct position. For example, movement of the cover from its correct position on the surgical treatment tool may affect the insulation performance.

It is an aspect to provide a novel technology that makes a cover less likely to come off a surgical tool such as a forceps device or makes the cover less likely to move from its correct position.

According to some embodiments, a forceps device may include a distal end part including a treatment part for treating an object; a columnar member connected to the distal end part; a pipe connected to the columnar member on a side thereof opposite the side with which the distal end part is connected; a cover mounted over the distal end part to the columnar member; and an enlarged-diameter portion having a larger diameter than an end face, on a side closer to the pipe, of the cover mounted over the columnar member. The enlarged-diameter portion may be located at a position covering the end face.

According to this aspect, the end of the trocar hardly gets caught on the end face of the cover when the forceps device is being removed from or inserted into the trocar. In addition, even if the end of the trocar comes in contact with the cover, the contact position can be shifted from the end face of the cover toward the distal end side.

In some embodiments, the enlarged-diameter portion may have a tapered shape with its diameter decreasing from an end adjacent to the columnar member toward an end adjacent to the pipe. This configuration makes the end of the trocar less likely to get caught on the end face of the cover when the forceps device inserted in the trocar is being removed. In addition, the angle between the axis of the trocar and the axis of the forceps device is an acute angle when the forceps device inserted in the trocar is being removed.

In some embodiments, a diameter of the enlarged-diameter portion at the end of the tapered shape adjacent to the pipe may be smaller than a diameter of the pipe. As a result, the end of the trocar is less likely to get caught on the end, which is adjacent to the pipe, of the enlarged-diameter portion when the forceps device inserted in the trocar is being removed.

In some embodiments, the enlarged-diameter portion may be provided at the columnar member or the pipe. This configuration makes the end of the trocar less likely to get caught on the end face of the cover around the region between the columnar member and the pipe.

In some embodiments, the diameter of the enlarged-diameter portion on the columnar member side may be smaller than an inner diameter of a trocar into which the forceps device is inserted. This configuration enables the forceps device to be inserted into the trocar.

In some embodiments, a wire for causing the treatment part to operate may be included. The columnar member may have a through-hole through which the wire extends.

Various embodiments here may include any combination of the components described above. Additionally, a method, a device, a system, and the like consistent with the above embodiments is included as aspects of the present disclosure.

According to various embodiments, a cover hardly comes off a forceps device.

Various embodiments will now be described with reference to the drawings. Components, members, and processes that are the same as or equivalent to each other illustrated in the drawings are represented by the same reference numerals, and redundant explanation will not be repeated where appropriate for conciseness. The disclosure is not intended to be limited to the various embodiments described herein, but are provided as examples, and any feature or any combination of features described in the embodiments is not necessarily essential.

### [Forceps Device]

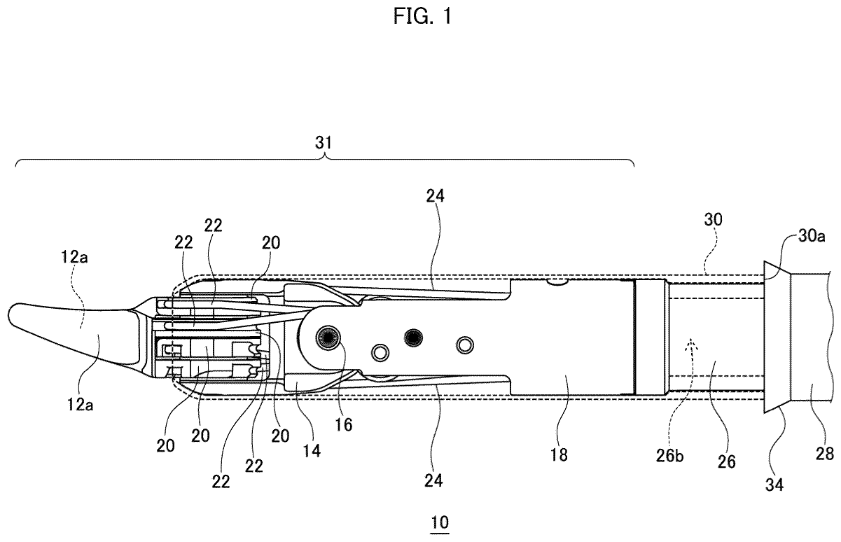

FIG. 1 is a front view of a forceps device according to an embodiment. The forceps device 10 for medical use illustrated in FIG. 1 includes a pair of grasping portions 12a and 12b, a support 14 that holds the pair of grasping portions 12a and 12b, a first rotating shaft 16 that turnably supports the support 14, a base member 18 that holds the first rotating shaft 16, four guide pulleys (not illustrated) arranged coaxially with the first rotating shaft 16, four jaw pulleys 20 supported coaxially with the pair of grasping portions 12a and 12b, four wires 22 running over the four guide pulleys and the four jaw pulleys 20, and two wires 24 for turning the support 14 about the first rotating shaft 16.

The base member 18 is connected with a front end of a base cap 26, which is a columnar member. A rear end of the base cap 26 is connected with a pipe 28. The forceps device 10 according to the embodiment is covered with a joint cover 30 having a cylindrical shape from the support 14 to the rear end of the base cap 26. Thus, foreign substances hardly enter gaps between components such as wires and pulleys during surgery. Note that the joint cover 30 may cover part of the grasping portions 12a and 12b in addition to the support 14. In some embodiments, for a forceps device that transmits electricity to the grasping portions 12a and 12b for hemostasis of patient's tissue, an insulating joint cover 30 may be used.

The base cap 26 is a plastic part having a cylindrical shape (columnar shape) and made of an engineering plastic material or the like. This configuration achieves both the strength and the insulation property of the part. Specifically, examples of the material include, but are not limited to, polyetheretherketone (PEEK) or polyetherimide (PEI).

For the pipe 28, such a material as carbon fiber reinforced plastic (CFRP) or glass fiber reinforced plastic (GFRP) is used, for example. Examples of the material for the joint cover 30 include, but are not limited to, butyl rubber, silicone rubber, and/or polyethylene.

As described above, the forceps device 10 may include a distal end part 31 including the grasping portions 12a and 12b, which constitute a treatment part for treating an object (a body site) to be operated, the base cap 26 connected to the distal end part 31, the pipe 28 connected to the base cap 26 on a side thereof opposite the side with which the distal end part 31 is connected, and the joint cover 30 mounted over the distal end part 31 and the base cap 26.

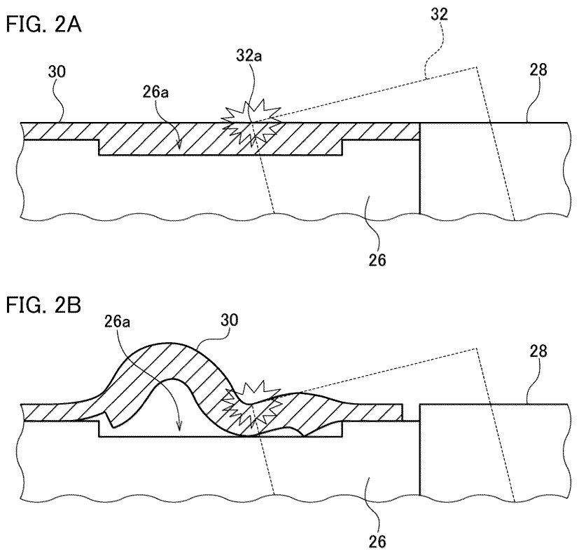

FIGS. 2A and 2B are schematic views for explaining a state in which the joint cover comes off the base cap, according to some embodiments. The forceps device 10 is inserted into a patient's body through a trocar 32 in laparoscopic surgery. The inserted forceps device 10 may incline or slide inside a patient's body. Thus, part of the joint cover 30 being caught on an end 32a of the trocar 32 (FIG. 2A) rises up at a recess 26a of the base cap 26 (FIG. 2B) as the forceps device 10 moves in a direction in which the forceps device 10 is removed from the trocar 32. As a result, an area of contact between the joint cover 30 and the recess 26a of the base cap 26 decreases, which reduces the friction therebetween, and therefore makes the joint cover 30 easier to be shifted relative to the base cap 26.

If a force is further applied to the joint cover 30 in this risen-up state from the trocar 32, the joint cover 30 may eventually come off the forceps device 10. The forceps device 10 according to some embodiments has an enlarged-diameter portion (large-diameter portion) having a larger diameter than the pipe 28 and the base cap 26, as will be described further below. For example, in some embodiments, the enlarged-diameter portion may be a hood that flairs out beyond an end of the base cap 26.

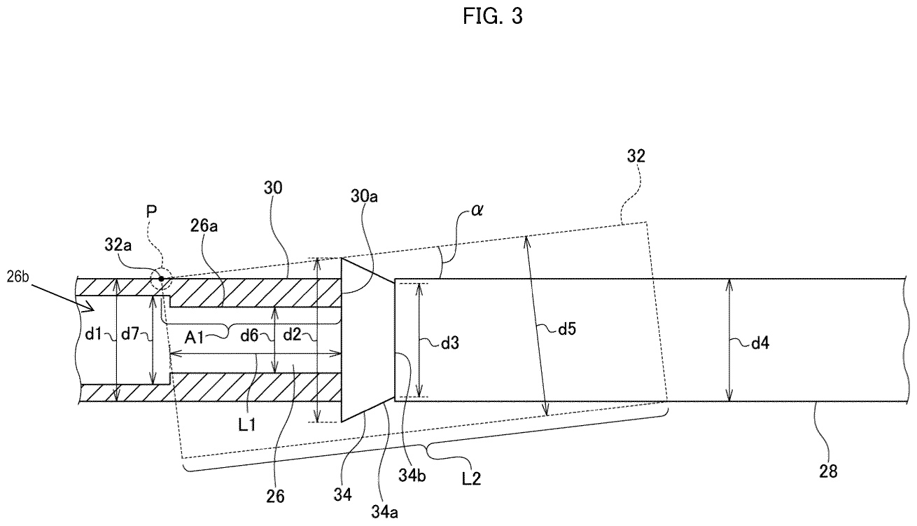

FIG. 3 is an enlarged view of a structure around the enlarged-diameter portion of the forceps device according to some embodiments. As illustrated in FIG. 3, an enlarged-diameter portion 34 has a diameter d2 that is larger than a diameter d1 of the joint cover 30 at an end face 30a, which is on a side closer to the pipe, of the joint cover 30 mounted over the base cap 26. According to some embodiments, the diameter d1 is set within a range from 4.0 to 15.0 mm (or advantageously within a range of 8.7+1.5 mm), and the diameter d2 is set within a range from 4.1 to 15.1 mm (or advantageously within a range of 9.6+1.5 mm). In some embodiments, the enlarged-diameter portion 34 may be located at a position covering the end face 30a. In other words, the enlarged-diameter portion 34 located at a position covering the end face 30a can hide the end face 30a as viewed from the end 32a of the trocar 32 when the forceps device 10 inserted in the trocar 32 is being removed.

The enlarged-diameter portion 34 provided as described above makes the end 32a of the trocar 32 less likely to get caught on the end face 30a of the joint cover 30 when the forceps device 10 is being removed from the trocar 32 or inserted into the trocar 32.

In some embodiments, the enlarged-diameter portion 34 may have a tapered shape 34a with its diameter decreasing from the base cap 26 side toward the pipe 28 side. As a result, the angle α between an axis of the trocar 32 and an axis of the forceps device 10 when the forceps device 10 inserted in the trocar 32 is being removed is an acute angle. Consequently, if the end 32a of the trocar 32 comes in contact with the joint cover 30, the contact position P can be shifted from the end face 30a of the joint cover 30 toward the distal end side.

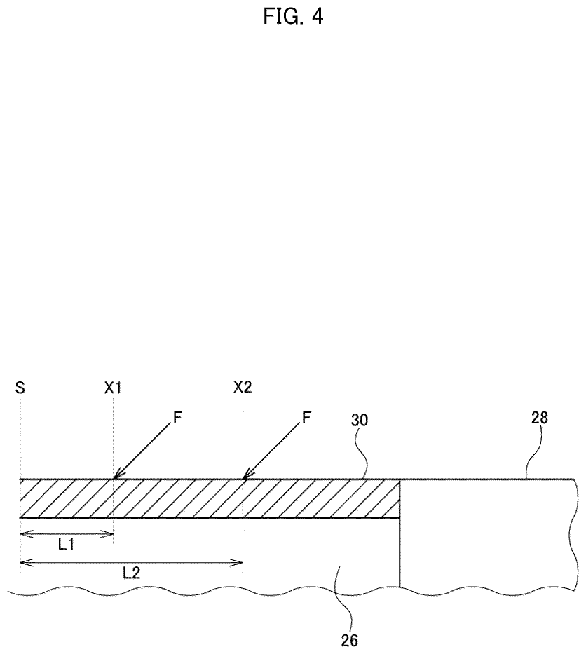

FIG. 4 is a schematic view for explaining the difference in deflection depending on the position at which the end of the trocar comes in contact with the joint cover, according to various embodiments. As illustrated in FIG. 4, a contact point X1 is at a distance L1 from a reference position S on the distal end side, and a contact point X2 is at a distance L2 (L2>L1) from the reference position S on the distal end side. In this case, the joint cover 30 is more stiff at the contact point X2. When the contact position P is not at the end face 30a of the joint cover 30 but is moved toward the distal end side as described above, the joint cover 30 is less easily bent at least at a part A1 (see FIG. 3), which is on the side closer to the pipe 28 with respect to the contact position P. As a result, the joint cover 30 is less likely to rise up and come off the base cap 26 due to static frictional force at the part A1.

Returning to FIG. 3, a diameter d3 of an end 34b, which is adjacent to the pipe, of the tapered shape 34a of the enlarged-diameter portion 34 may be smaller than a diameter d4 of the pipe 28. In some embodiments, the diameter d3 may be within a range from 3.9 to 14.9 mm (or advantageously within a range of 8.0±1.5 mm), and the diameter d4 may be within a range from 4.0 to 15.0 mm (or advantageously within a range of 8.4±1.5 mm). As a result, the end 32a of the trocar 32 is less likely to get caught on the end 34b, which is adjacent to the pipe, of the enlarged-diameter portion 34 when the forceps device 10 inserted in the trocar 32 is being removed.

In some embodiments, the enlarged-diameter portion 34 may be provided as part of the base cap 26, part of the pipe, or a separate component in the forceps device 10. This structural flexibility makes the end 32a of the trocar 32 less likely to get caught on the end face 30a of the joint cover 30 around the region between the base cap 26 and the pipe 28.

In some embodiments, the diameter d2 of the enlarged-diameter portion 34 on the base cap 26 side may be smaller than an inner diameter d5 of the trocar into which the forceps device 10 is inserted. This configuration enables the forceps device 10 to be inserted into the trocar 32. According to an embodiment, the inner diameter d5 may be within a range from 5.0 to 15.0 mm (or advantageously within a range of 10 to 15 mm), and the diameter d2 of the enlarged-diameter portion 34 may be within a range of 99% of the inner diameter d5 of the trocar 32 or smaller. In some embodiments, the effective length L2 of the trocar 32 may be within a range from 30 to 200 mm (or advantageously within a range of 30 to 100 mm).

If the diameter d2 is too large, the change in the posture of the forceps device 10 relative to the trocar 32 is too small. In contrast, if the diameter d2 is too small, the inclination a of the trocar 32 relative to the forceps device 10 tends to be large, and the amount (contact load) by which the end 32a of the trocar 32 bites into the joint cover 30 at the contact position P becomes larger. Furthermore, the contact position P becomes likely to be closer to the end face 30a of the joint cover 30. The position at which the enlarged-diameter portion 34 is provided and the diameter d2 are therefore advantageously set so that the inclination a relative to the trocar 32 into and from which the forceps device 10 is inserted and removed is an acute angle.

Note that the enlarged-diameter portion 34 supporting an inner wall of the trocar 32 partly bears the load applied when the trocar 32 comes in contact with the joint cover 30. This configuration makes the amount by which the end 32a of the trocar 32 bites into the joint cover 30 smaller. The presence of the enlarged-diameter portion 34 enables the position at which the end 32a of the trocar 32 comes into contact with the joint cover 30 to be shifted toward the distal end side with respect to the end face 30a of the joint cover 30. As a result, even if the trocar 32 comes into contact with the joint cover 30, the trocar 32 touches a position X1, which is closer to the distal end side and has a higher stiffness, rather than a position X2, which is closer to the end face 30a of the joint cover 30 and has a lower stiffness (see FIG. 4). This configuration prevents or minimizes deflection of the joint cover 30.

Furthermore, when the trocar 32 comes in contact with the position X2, the friction between the base cap 26 and the joint cover 30 generated over an area from the position X2 to the end face 30a acts as a reaction force. Thus, when the trocar 32 comes in contact with the position X1, which is closer to the distal end side than the position X2, the friction between the base cap 26 and the joint cover 30 generated over the area from the contact position to the end face 30a is larger, which makes the joint cover 30 less likely to come off.

According to some embodiments, the base cap 26 may have a through-hole 26b through which wires 22 and wires 24 for causing the grasping portions 12a and 12b, which constitute the treatment part, to perform such operation as opening/closing and turning. The length L1 of the recess 26a formed on a portion of the base cap 26 close to the pipe 28 may be within a range of 7.0±3 mm, and a diameter d6 of a small-diameter portion of the recess 26a may be within a range of 3.0 to 11.9 mm (or advantageously within a range of 7.2±1.5 mm). In some embodiments, a diameter d7 of a large-diameter portion of the base cap 26 may be within a range of 3.1 to 12.0 mm (or advantageously within a range of 7.8±1.5 mm).

Furthermore, the diameter d2 of the enlarged-diameter portion 34 may be advantageously set so that the contact position P at which the end 32a of the trocar 32 comes into contact with the joint cover 30 is on the side close to the distal end side with respect to the recess 26a of the base cap 26. In this case, part of the joint cover 30 fitted in the recess 26a close to the pipe 28 with respect to the contact position P is caught by the stepped portion, which makes the joint cover 30 still less likely to come off the base cap 26.

Note that the enlarged-diameter portion 34 may be a plastic part made of a material such as an engineering plastic material. This configuration achieves both the strength and the insulation property of the part. Specifically, examples of the material include, but are not limited to, polyetheretherketone (PEEK) or polyetherimide (PEI).

While an example of a forceps device having an annular recess 26a around the outer circumference of the base cap 26 has been described above, embodiments are not limited thereto and, in some embodiments, a forceps device without the recess 26a, that is, with no stepped portion corresponding to a side wall of the recess 26a may also applicable.

While various embodiments have been described above with reference to the drawings, the embodiments are not limited to those described above, and any combination or substitution of components is included in the present disclosure. In addition, modifications such as combinations, changes in the order of processes, and various changes to the various embodiments may be made on the basis of knowledge of a person skilled in the art, and such modified embodiments are included within the scope of the appended claims.

What is claimed is:

1. A forceps device comprising:
a distal end part including a treatment part for treating an object;
a columnar member connected to the distal end part;
a pipe connected to the columnar member on a side thereof opposite a side with which the distal end part is connected;
a cover mounted over the distal end part and the columnar member; and
an enlarged-diameter portion having a larger diameter than an end face, on a side closer to the pipe, of the cover that is mounted over the columnar member,
wherein the enlarged-diameter portion is located at a position covering the end face.

2. The forceps device according to claim 1,
wherein the enlarged-diameter portion has a tapered shape in which a diameter of the enlarged-diameter portion decreases from an end adjacent to the columnar member toward an end adjacent to the pipe.

3. The forceps device according to claim 2,
wherein a diameter of the enlarged-diameter portion at the end adjacent to the pipe is smaller than a diameter of the pipe.

4. The forceps device according to claim 1,
wherein the enlarged-diameter portion is provided at the columnar member or at the pipe.

5. The forceps device according to claim 1,
wherein a diameter of the enlarged-diameter portion on a columnar member side is smaller than an inner diameter of a trocar into which the forceps device is configured to be inserted.

6. The forceps device according to claim 1, further comprising:
a wire connected to the treatment part for causing the treatment part to operate,
wherein the columnar member comprises a through-hole through which the wire extends.

7. A forceps device comprising:
a distal end part including a treatment part for treating an object;
a base cap connected proximally to the distal end part;
a pipe connected proximally to the base cap;
a cover that covers the distal end part and the base cap and has an end face at a proximal end of the base cap; and
an enlarged-diameter portion having a diameter that is larger than a diameter of the end face of the cover.

8. The forceps device according to claim 7,
wherein the enlarged-diameter portion has a tapered shape in which the diameter of the enlarged-diameter portion decreases from an end of the enlarged-diameter portion that is adjacent to the base cap toward an end of the enlarged-diameter portion that is adjacent to the pipe.

9. The forceps device according to claim 8,
wherein the diameter of the enlarged-diameter portion at the end of the enlarged-diameter portion that is adjacent to the pipe is smaller than a diameter of the pipe.

10. The forceps device according to claim 7,
wherein the enlarged-diameter portion is provided at the base cap or at the pipe.

11. The forceps device according to claim 7,
wherein the diameter of the enlarged-diameter portion is smaller than an inner diameter of a trocar into which the forceps device is configured to be inserted.

12. The forceps device according to claim 7, further comprising:
a wire connected to the treatment part,
wherein the base cap comprises a through-hole through which the wire extends.

13. A forceps device comprising:
a distal end part including a treatment part for treating an object;
a base cap connected proximally to the distal end part;
a pipe connected proximally to the base cap;
a cover that covers the distal end part and the base cap and has an end face at a proximal end of the base cap; and
a hood having a diameter that is larger than a diameter of the end face of the cover.

14. The forceps device according to claim 13,
wherein the hood has a tapered shape in which a diameter of the hood decreases from a first end of the hood that is adjacent to the base cap toward a second end of the hood that is adjacent to the pipe.

15. The forceps device according to claim 14,
wherein a diameter of hood at the second end is smaller than a diameter of the pipe.

16. The forceps device according to claim 13,
wherein the hood is mounted at the base cap or at the pipe.

17. The forceps device according to claim 13,
wherein a diameter of the hood is smaller than an inner diameter of a trocar into which the forceps device is configured to be inserted.
